(19) United States
(12) Patent Application Publication
(10) Pub. No.: US 2009/0294549 A1
(43) Pub. Date: Dec. 3, 2009

(54) FRAGRANCE DELIVERY SYSTEM

(76) Inventor: Bruce M. Campbell, Mattituck, NY (US)

Correspondence Address: LILLIE LAW, LLC, 269 SOUTHPORT ST, RONKONKOMA, NY, 11779, US

(21) Appl. No.: 12/127,322

(22) Filed: May 27, 2008

(51) Int. Cl. A61L 9/04; A61L 9/00

(52) U.S. Cl. 239/44; 239/145

(57) ABSTRACT

A passive fragrance delivery system having: a longitudinal body having a proximal and distal end, a diameter and length, with an inner core and outer sheath substantially of the same length and having an axis extending vertically there through; the inner core made from a material that is substantially rigid and substantially provides a structure for the outer sheath; wherein the body has the characteristic which permits capillary action of a liquid when one end of the body is place into a liquid source, wherein the second end within a predetermined period becomes occupied with the liquid source; the body further providing emission of the liquid source between the ends.

### CROSS REFERENCE TO RELATED APPLICATION(S)

This application claims priority from U.S. Pat. App. No. 60/940,0124 entitled Super Fragrance Stick filed on 25 May 2007, wherein the current application expressly incorporates by reference the full spirit and scope of the prior applications.

### BACKGROUND OF THE INVENTION

This invention relates to a passive fragrance delivery system, in particular, a system that provides a fragrance delivery system without the need of combustion, electricity, or secondary heat sources hence providing an infinite life expectancy of the same.

The current technology are essentially limited to sticks fabricated from bamboo, rattan, reeds, wood, etc., which are then placed in a jar of liquid fragrance and they are supposed to diffuse the fragrance into the room. The main issue with this current technology is that all of these materials do not support very efficient capillary action so the fragrance is only traceably noticeable, and the liquid will sit in the jar for 6 months and just look like it does something, but you practically are unable to discover the scent unless your within inches of the source. The existing product(s) look the part but fails to provide a measurable amount of fragrance in a timely manner.

The present invention can utilize any and all of those existing reeds, no matter what the material, but is preferably a non-oxidizing, malleable material encased in a braided jacket made of cotton, rayon, et al, that promotes capillary action. The 2 types of Super Fragrance Sticks (aka SFS), in this case cotton and rayon, took between 1-2 hours to be fully saturated to the top and the fragrance delivery was immediate and intense. The Super Fragrance Sticks work within minutes to fragrance a large room without the need of combustion, electricity, or secondary heat sources. Furthermore, the present invention will be embodying all the colors possible that the Super Fragrance Sticks could be colored with for esthetic purposes. They can easily be dyed with fabric dye to any color to compliment the color of the fragrance or the consumer's home decor. Moreover, when left in their natural color, they become the color of the fragrance as it saturates up the stick. Within a minute the SFS goes from white to red, blue, or whatever colour is so desired. One can observe the fragrance travel up the stick so that you know its working.

Illustrated below in Table 1, is the comparison of using ten (10) ten and three quarter inch reeds (10¾″) for a given liquid source (fragrance), compared to four (4) ten and three quarter inch SFSs (10¾″) for an identical liquid source over a time period of 80 days. Moreover, illustrated in Table 2 is the equivalent results converted to ten (10) ten and three quarter inch SFSs (10¾″). This date are Illustrated in graph 1, clearly evidencing the superiority and infallibility of the present invention as compared to the performance of the prior art. In fact, a mere four (4) SFSs out perform ten (10) prior art reeds buy nearly sixteen percent (16%) by using some sixty (60%) less material. When converted to equivalent quantity of rods (10) having the equivalent length (10.75 inches) for the equivalent time period (80 days), the present invention outperforms the prior art on an average of 319% per period.

TABLE 1

| | Apple Mac w\ 10 - | | | Apple Mac w\ 4 - | | | |
|---|---|---|---|---|---|---|---|
| | 10.75″ Reeds | | | 10.75″ SFS | | | |
| Period/Days | Weight | Loss | Loss/Period | Weight | Loss | Loss/Period | % diff/periods |
| 0 | 357.61 | 0.00 | | 345.95 | 0.00 | | |
| 1 | 352.26 | 5.35 | 5.35 | 336.55 | 9.40 | 9.40 | 75.70% |
| 2 | 349.16 | 8.45 | 3.10 | 331.29 | 14.66 | 5.26 | 69.68% |
| 3 | 346.27 | 11.34 | 2.89 | 326.70 | 19.25 | 4.59 | 58.82% |
| 4 | 342.92 | 14.69 | 3.35 | 322.04 | 23.91 | 4.66 | 39.10% |
| 7 | 336.60 | 21.01 | 6.32 | 313.03 | 32.92 | 9.01 | 42.56% |
| 9 | 333.24 | 24.37 | 3.36 | 307.49 | 38.46 | 5.54 | 64.88% |
| 14 | 325.01 | 32.60 | 8.23 | 295.64 | 50.31 | 11.85 | 43.99% |
| 18 | 318.83 | 38.78 | 6.18 | 285.50 | 60.45 | 10.14 | 64.08% |
| 22 | 313.83 | 43.78 | 5.00 | 277.38 | 68.57 | 8.12 | 62.40% |
| 28 | 305.79 | 51.82 | 8.04 | 264.68 | 81.27 | 12.70 | 57.96% |
| 34 | 300.51 | 57.10 | 5.28 | 254.99 | 90.96 | 9.69 | 83.52% |
| 36 | 298.57 | 59.04 | 1.94 | 251.51 | 94.44 | 3.48 | 79.38% |
| 38 | 296.78 | 60.83 | 1.79 | 248.27 | 97.68 | 3.24 | 81.01% |
| 44 | 292.82 | 64.79 | 3.96 | 239.76 | 106.19 | 8.51 | 114.90% |
| 45 | 292.26 | 65.35 | 0.56 | 238.47 | 107.48 | 1.29 | 130.36% |
| 50 | 287.99 | 69.62 | 4.27 | 232.74 | 113.21 | 5.73 | 34.19% |
| 52 | 286.81 | 70.80 | 1.18 | 231.01 | 114.94 | 1.73 | 46.61% |
| 60 | 282.97 | 74.64 | 3.84 | 224.79 | 121.16 | 6.22 | 61.98% |
| 67 | 279.23 | 78.38 | 3.74 | 218.69 | 127.26 | 6.10 | 63.10% |
| 73 | 276.82 | 80.79 | 2.41 | 214.44 | 131.51 | 4.25 | 76.35% |
| 80 | 274.16 | 83.45 | 2.66 | 209.93 | 136.02 | 4.51 | 69.55% |

| | | | | |
|---|---|---|---|---|
| Overall % loss | 23.34% | 39.32% | | |
| Difference | | 15.98% | | |
| # of reeds | 10.00 | 4 | | |
| Length/reed | 10.75 | 10.75 | | |
| Overall length | 107.50 | 43 | 150.00% | |
| | | | −60.00% | 4 SFS did it with −60% less |
| | | | | material but yet yielded a −16% |
| | | | | improvement. |
| loss/reed | 8.35 | 34.01 | 307.49% | increase! |

TABLE 2
*Estimated equivalent

| | “Apple Mac | | | |
|---|---|---|---|---|
| | w\ 10-10.75″ SFS | | | |
| Period/Days | Weight | Loss | Loss/Period | % diff/period |
| 0 | 345.95 | 0.00 | | |
| 1 | 336.55 | 23.50 | 23.50 | 339.25% |
| 2 | 331.29 | 36.65 | 13.15 | 324.19% |
| 3 | 326.70 | 48.13 | 11.48 | 297.06% |
| 4 | 322.04 | 59.77 | 11.65 | 247.76% |
| 7 | 313.03 | 82.30 | 22.53 | 256.41% |
| 9 | 307.49 | 96.15 | 13.85 | 312.20% |
| 14 | 295.64 | 125.78 | 29.63 | 259.96% |
| 18 | 285.50 | 151.13 | 25.35 | 310.19% |
| 22 | 277.38 | 171.43 | 20.30 | 306.00% |
| 28 | 264.68 | 203.18 | 31.75 | 294.90% |
| 34 | 254.99 | 227.40 | 24.23 | 358.81% |
| 36 | 251.51 | 236.10 | 8.70 | 348.45% |
| 38 | 248.27 | 244.20 | 8.10 | 352.51% |
| 44 | 239.76 | 265.48 | 21.28 | 437.25% |
| 45 | 238.47 | 268.70 | 3.22 | 475.89% |
| 50 | 232.74 | 283.03 | 14.33 | 235.48% |
| 52 | 231.01 | 287.35 | 4.33 | 266.53% |
| 60 | 224.79 | 302.90 | 15.55 | 304.95% |
| 67 | 218.69 | 318.15 | 15.25 | 307.75% |
| 73 | 214.44 | 328.78 | 10.63 | 340.87% |
| 80 | 209.93 | 340.05 | 11.28 | 323.87% |
| | | | Average = | 319.06% |

### SUMMARY OF THE INVENTION

The present invention is directed to passive fragrance delivery system having a primary advantage of providing a green energy delivery.

Another advantage of the present disclosure includes providing a delivery system having a pragmatic and near infinite life expectancy.

A still further advantage of the present disclosure includes providing a design that provides increased performance in shortened delivery time, greater range of delivery, greater duration of delivery, and increased reliability, thereby yielding a superior quality product and product yield.

An additional advantage of the present disclosure includes providing a delivery system which further includes aesthetic colourization, which indicates the progress of the liquid saturation.

Another advantage of the present disclosure includes providing a delivery design that not only is pleasant to the sense of smell, and sight, but extends to the increased gratification of the sense of touch, for example, this technology may be use with citronella as a liquid source thereby minimizing if not eliminating the presence of undesirable insects and pests.

Additionally, the current disclosure provides a flip-less, yet flippable body, that does not exhibit the permeability problems of the prior technologies, thereby providing a near maintenance free delivery system.

An additional advantage of the present disclosure includes an infinite amount of poseable positions afforded by the malleably, reusealable design having a memory absent substantial distortion, and interatively so.

Other advantages, advantages, and novel features of the present invention will become more apparent from the following detailed description when taken in conjunction with the accompanying drawings.

### BRIEF DESCRIPTION OF THE DRAWING(S)

The following drawings, in which like reference characters indicate like parts, are provided for illustration of the invention and are not intended to limit the invention in any manner whatsoever.

FIG. 1 illustrates the prior art;

FIG. 2 illustrates a preferred embodiment of the present invention as deployed;

FIG. 2A illustrates the sheath about the inner core;

FIG. 3 illustrates the preferred embodiment having an optional aesthetic device, namely, a holiday decoration;

FIG. 3A illustrates the bottom view of the optional aesthetic device illustrated in FIG. 3;

FIG. 4 illustrates the preferred embodiment having an alternative optional aesthetic devices, namely, a flower top and leaves; and

FIG. 5 illustrates a plurality of aesthetic devices of FIG. 4.

### DETAILED DESCRIPTION OF THE INVENTION

The following descriptions of the preferred embodiments are presented to illustrate the present invention and are not to be construed to limit the claims in any manner whatsoever.

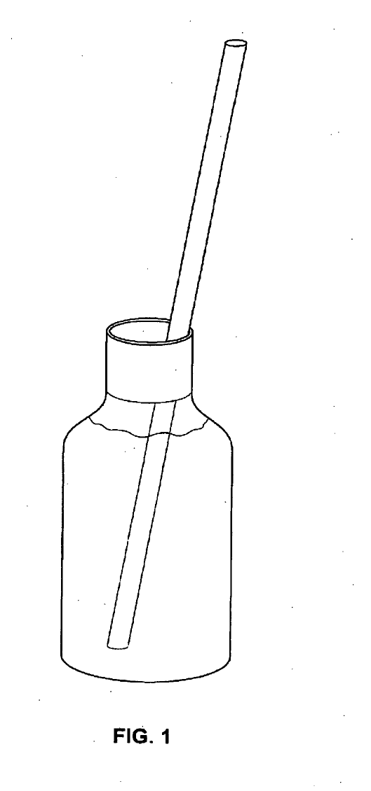

FIG. 1 illustrates the prior art, wherein a simple rattan reed is immersed in a fragrance solution within a liquid receptacle.

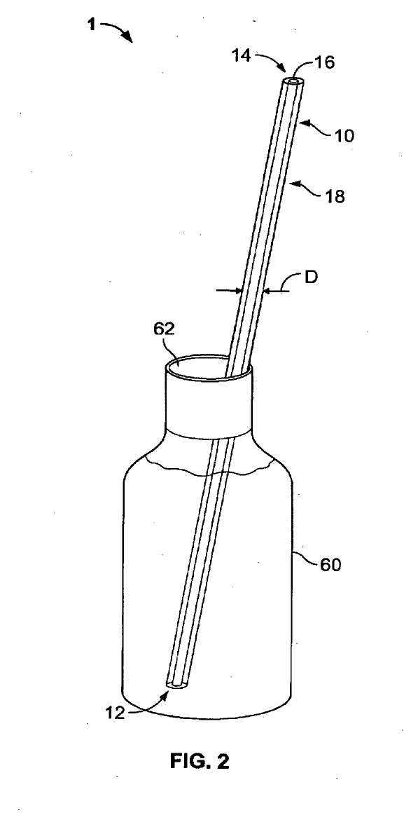

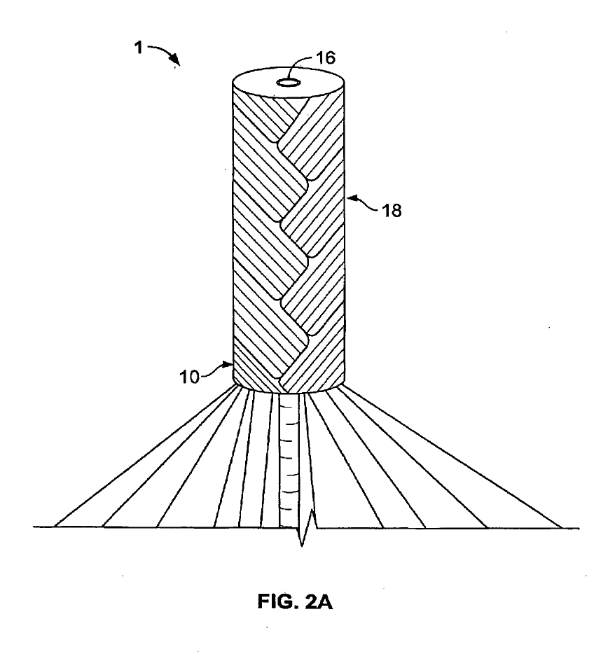

FIG. 2 illustrates a preferred embodiment of the present invention, which is directed to a passive fragrance delivery system 1 comprising: a longitudinal body 10 having a proximal and distal end 12,14 a diameter D and a length L, with an inner core 16 and outer sheath 18 substantially of the same length and having an axis extending vertically there through; said inner core 16 is made from a material that is substantially rigid and substantially provides a structure for the outer sheath 18 wherein said body has the characteristic which permits capillary action of a liquid when one end 12 of the body 10 is place into a liquid source, such as a fragrance with/without a colour factor, wherein the second end 14, within a predetermined period, becomes occupied with the liquid source via capillary action; said body 10 further providing emission of the liquid source about its outer surface. In practice, the body 10 is flippable, that is, the body 10 may be inverted so that the distal end 14 is submerged into the liquid source, and the proximal end 12 is no longer, however, it is not required because the system 1 does not clog, unlike the prior art.

In the preferred embodiment, the inner core 16 is made of a material that is non-oxidizing, such as aluminum wire, however, the essence of the inner core 16 is multifaceted, first, it provides a spine for the outer sheath 18, second, it is made of a malleably reusable material that is capable of deformation absent substantial distortion such that the core 16 may be manipulated into various poses, and/or positions, and iteratively so. The inner core 16 may be of various diameters, however, it is preferred that the inner core 16 have less than or equal to about 1.0 to 3.0 mm, more preferably less than or equal to about 1.5 mm, although it is envisioned that in alternative embodiments, this range maybe from about 1/16 of an inch to about 1 inch.

The outer sheath 18 is preferably fabricated from an organic material, e.g. cotton, as organic functions superiorly to inorganic, and is naturally renewable (green), although it is envisioned that other materials may be incorporated, either in whole or in part, such as rayon. Moreover, the material incorporated is selected to promote capillary action and may either be colourfast, non-colourfast, or both, either in whole or in part, that is, the material can be selectively chosen to maintain its original colour (colourfast) and/or to selectively change its colour (non-colourfast) to a certain degree to a desired colour so as to evidence the capillary action and/or for aesthetic purposes. For example, a white, non-colourfast cotton is deployed into a solution, eventually, the cotton sheath 18 will wick the solution and change colour thereby evidencing the presence of the liquid source at any given point and hence whether the system 1 is working (i.e., evaporation of the solution) and also to reflect the desired colour as defined by the solution, for example, a red solution designed to change the colour of the white, non-colourfast cotton sheath 18 to red. Moreover, if the non-colourfast sheath 18 was yellow, a red solution would change the sheath 18 to orange. Furthermore, the sheath 18 itself may have a plethora of designs, and colours, for example, single coloured, or multicoloured, either with or without patterns thereof, an example thereof is illustrated in FIG. 2A, wherein the sheath 18 is woven about the inner core 16.

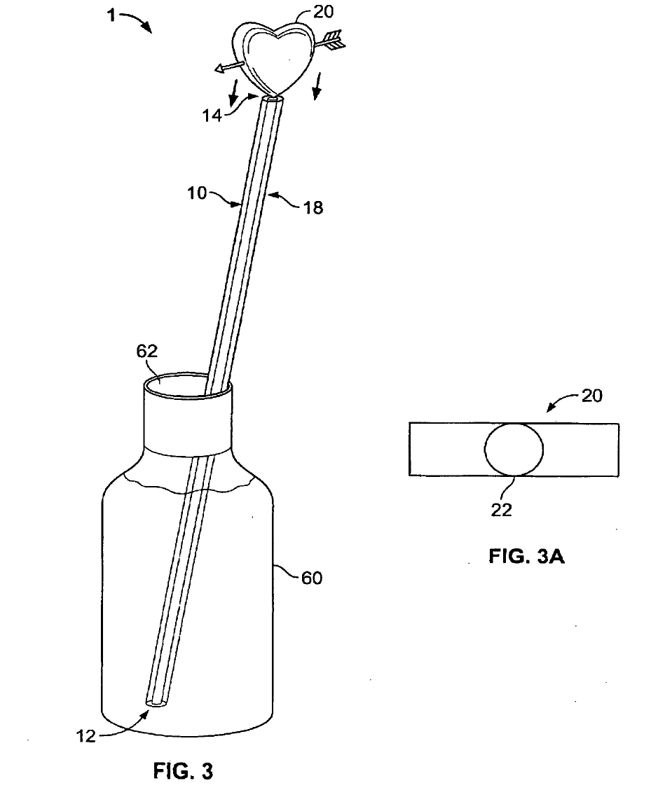

FIG. 3 illustrates the preferred embodiment having an optional aesthetic device or topper 20 that is interchangeable, for example, for the given holiday season, such as, a heart for Valentine's day, a shamrock for St. Patrick's day, an egg for Easter, etc. The aesthetic device 20 may be fabricated from either a permeable or non-permeable material or both, wherein, a wicking barrier 22 resides in between the aesthetic device 20 and the distal end 14 of the longitudinal body 10 providing means for preventing alteration of the colour of the aesthetic device 20 derived from the liquid source, although, if the aesthetic device 20 is made from a colourfast material, the wicking barrier 22, in this scenario, would only be necessary if it is desired that the device 20 be excluded from the evaporation process, whereas if the device 20 was fabricated from a non-colourfast material, then this would be an additional basis for excluding the device 20 from the evaporation process if this is so desired by the end user, although, it is up to the end user whether not the wicking feature should be implemented. Nonetheless, the wicking barrier 22 comprises a sealant, best illustrated in FIG. 3A, a bottom view of the device 20. In the preferred embodiment, the wicking barrier is either a physical and/or a chemical additive/layer applied to the orifice 21 in the bottom of the device 20. For example, a physical insert of three-quarter (¾″) inch long piece of polypropylene (plastic) tubing with one end sealed closed having an inside diameter slightly larger than the diameter of the outer sheath 18.

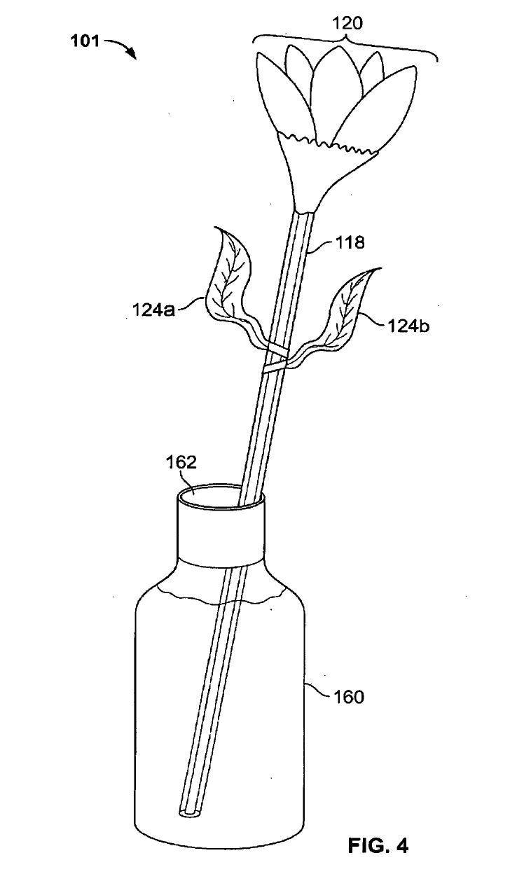

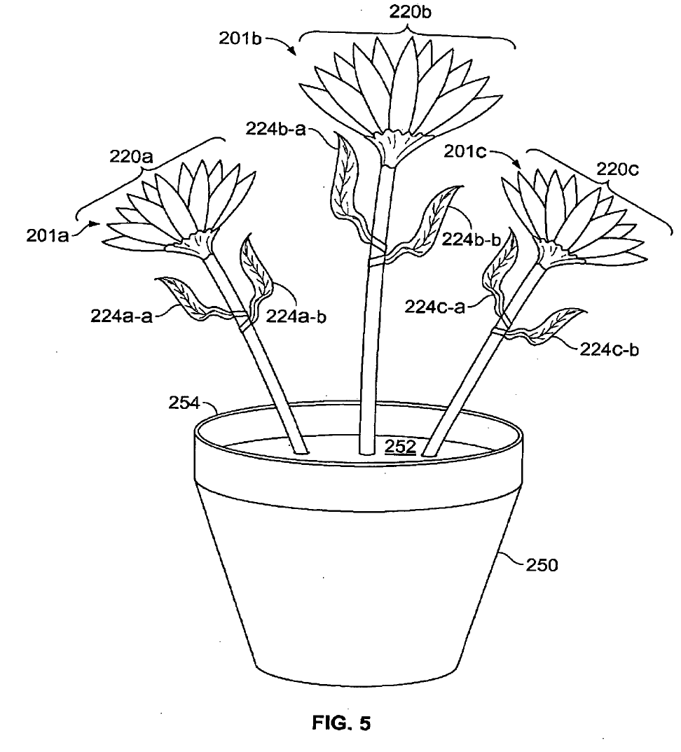

FIG. 4 illustrates an alternative embodiment, here a flower 101, wherein the flower head 120 comprises, inter alia, the corolla, sepals, pistil, stamen, ovules. The head 120 may be comprised of material either similar or different than the outer sheath 118, but at least similar to the extent that provide either permeable or non-permeable material for evaporation and wicking of the liquid. It is envisioned that a plurality of these flowers 101 may be incorporated into an arrangement, as illustrated in FIG. 5, wherein the flowers 201a, 201b, 201c may either be the same or different. For example, one may be a tiger lily having a certain colour and scent, wherein a second, e.g., a daisy, marigold, tulip, rose, has a certain colour and scent, wherein each flower 201a-c in this case has a different liquid source thereby providing a different scent and/or colour. In this regard, various synthetic arrangements may be fabricated/provided without watering.

Moreover, the system 101 may further include at least one leaf 124 so as to better simulate a real flower. If a plurality of leaves 124 is desired for a given longitudinal body 110 (which is analogous to the stem of a flower), each leaf 124a, 124b may be of the same or different size and orientation. Each 124a, 124b may be either permeable material for evaporation and wicking of the liquid or not, wherein the implementation of either is a function of the cubic area of the room, convection, strength of the fragrance selected, and the individual's personal preference of their target ambiance. Each leaf 124 is releasably adjustable vertically about the body 110 and rotationally as well until in the desired position.

All the above embodiments may be sold individually or together, either in whole or in part, as a kit, wherein the end user may mix and match the parts for a plethora of reasons, for example, the holiday season. For example, the kit may include an outer container 250 having an upper surface 252 with a perimeter 254, which looks like a flower pot, or any other desirable container, configured and dimensioned to receive a plurality of smaller liquid receptacles 260. The smaller receptacles 260 holding a liquid source with an orifice 162 near the top so to provide access to the liquid source by the body 210. In this particular embodiment, only one flower 201 is inserted into each receptacle 260, however, it is envisioned that a plurality of flowers 201 (not shown) may be used in each receptacle 260. Moreover, once the desired quantity of flowers 201 are inserted, then a synthetic grass, or moss like covering (not shown) can be placed above the openings 262 so as to camouflage the interface such that the entire upper surface 252 is covered to the perimeter 254.

All of the above referenced patents; patent applications and publications are hereby incorporated by reference. Many variations of the present invention will suggest themselves to those of ordinary skill in the art in light of the above detailed description. All such obvious modifications are within the full-intended spirit and scope of the claims of the present application both literally and in equivalents recognized at law.

What is claimed is:

1. A passive fragrance delivery system comprising:
a longitudinal body having a proximal and distal end, a diameter and length, with an inner core and outer sheath substantially of the same length and having an axis extending vertically there through; said inner core made from a material that is substantially rigid and substantially provides a structure for the outer sheath; wherein said body has the characteristic which permits capillary action of a liquid when one end of the body is place into a liquid source, wherein the second end within a predetermined period becomes occupied with the liquid source; said body further providing emission of the liquid source between said ends.

2. A passive fragrance delivery system as in claim 1, wherein the inner core is a non-oxidizing material.

3. A passive fragrance delivery system as in claim 1, wherein the inner core is a malleable material that is capable of deformation absent substantial distortion such that the core may be manipulated into a plurality of poses while having position memory.

4. A passive fragrance delivery system as in claim 1, wherein the outer sheath is fabricated from a material that is selected from the group consisting of: an organic, and inorganic material.

5. A passive fragrance delivery system as in claim 1, wherein the colourfastness of the material is selected from the group consisting of: colourfast, non-colourfast, or both.

6. A passive fragrance delivery system as in claim 1, wherein the colour of the sheath is selected from the group consisting of: monocoloured, and multicoloured, wherein the sheath may optionally have a pattern design.

7. A passive fragrance delivery system as in claim 1, further including an aesthetic device having an orifice in the bottom for receiving one end of the body, said device being fabricated from a material selected from the group consisting of: permeable, and non-permeable, or both; said device further optionally including a wicking barrier about the orifice.

8. A passive fragrance delivery system as in claim 7, wherein said barrier is selected from the group consisting of: a chemical layer, and a physical layer.

9. A passive fragrance delivery system as in claim 7, wherein said device is selected from the group consisting of a holiday emblem, a sporting device, a team emblem, a letter, a number, a symbol, and a flower; and optionally, a secondary aesthetic device configured and dimensioned to be releasably affixed about the body between said ends, and said secondary device being fabricated from a material selected from the group consisting of: permeable, and non-permeable, or both; wherein said material further has the characteristics of being malleably poseable with memory, said secondary device further optionally including a wicking barrier about the orifice, wherein said barrier is selected from the group consisting of: a chemical layer, and a physical layer.

10. A passive fragrance delivery system as in claim 9, wherein the secondary device includes a leaf.

11. A passive fragrance delivery system comprising:
a longitudinal body having a proximal and distal end, a diameter and length, with an inner core and outer sheath substantially of the same length and having an axis extending vertically there through; said inner core made from a material that is substantially rigid and substantially provides a structure for the outer sheath; wherein said body has the characteristic which permits capillary action of a liquid when one end of the body is place into a liquid source having both a scent and a colour contained in a liquid receptacle having an orifice near the top to provide access to the liquid source by the body, wherein the second end within a predetermined period becomes occupied with the liquid source; said body further providing emission of the liquid source between said ends.

12. A passive fragrance delivery system as in claim 11, wherein the inner core is a non-oxidizing material.

13. A passive fragrance delivery system as in claim 11, wherein the inner core is a malleable material that is capable of deformation absent substantial distortion such that the core may be manipulated into a plurality of poses while having position memory.

14. A passive fragrance delivery system as in claim 11, wherein the outer sheath is fabricated from a material selected from the group consisting of: an organic, and inorganic material; and the colour of the sheath is selected from the group consisting of: monocoloured, and multicoloured, wherein the sheath may optionally have a pattern design, and wherein the colourfastness of the material is selected from the group consisting of: colourfast, non-colourfast, or both.

15. A passive fragrance delivery system as in claim 11, further including an aesthetic device having an orifice in the bottom for receiving one end of the body, said device being fabricated from a material selected from the group consisting of: permeable, and non-permeable, or both; said device further optionally including a wicking barrier about the orifice.

16. A passive fragrance delivery system as in claim 15, wherein said barrier is selected from the group consisting of: a chemical layer, and a physical layer.

17. A passive fragrance delivery system as in claim 15, wherein said device is selected from the group consisting of a holiday emblem, a letter, a number, a sports device, a team emblem, a symbol, and a flower; and optionally, a secondary aesthetic device configured and dimensioned to be releasably affixed about the body between said ends, and said secondary device being fabricated from a material selected from the group consisting of: permeable, and non-permeable, or both; wherein said material further has the characteristics of being malleably poseable with memory, said secondary device further optionally including a wicking barrier about the orifice, wherein said barrier is selected from the group consisting of: a chemical layer, and a physical layer.

18. A passive fragrance delivery system as in claim 17, wherein the secondary device is one or more leaves.

19. A passive fragrance delivery system comprising:
a plurality of longitudinal bodies, each body having a proximal and distal end, a diameter and length, with an inner core and outer sheath substantially of the same length relative to the body, and having an axis extending vertically there through; said inner core made from a material that is substantially rigid and substantially provides a structure for the outer sheath; wherein said body has the characteristic which permits capillary action of a liquid when one end of the body is place into a liquid source having both a scent and a colour contained in a liquid receptacle having an orifice near the top to provide access to the liquid source by the body, wherein the second end within a predetermined period becomes occupied with the liquid source; said body further providing emission of the liquid source between said ends; wherein each body is placed selectively into its own liquid receptacle, and each receptacle is placed into a common outer container, said outer container being larger than the volume of all liquid receptacles, having an upper surface with a perimeter.

20. A passive fragrance delivery system as in claim 19, wherein the inner core is a non-oxidizing material; wherein the inner core is a malleable material that is capable of deformation absent substantial distortion such that the core may be manipulated into a plurality of poses while having position memory.

21. A passive fragrance delivery system as in claim 19, wherein the outer sheath is fabricated from a material selected from the group consisting of: an organic, and inorganic material; and the colour of the sheath is selected from the group consisting of: monocoloured, and multicoloured, wherein the sheath may optionally have a pattern design, and wherein the colourfastness of the material is selected from the group consisting of: colourfast, non-colourfast, or both.

22. A passive fragrance delivery system as in claim 19, further including an aesthetic device having an orifice in the bottom for receiving one end of the body, said device being fabricated from a material selected from the group consisting of: permeable, and non-permeable, or both; said device further optionally including a wicking barrier about the orifice.

23. A passive fragrance delivery system as in claim 22, wherein said barrier is selected from the group consisting of: a chemical layer, and a physical layer.

24. A passive fragrance delivery system as in claim 22, wherein said device is selected from the group consisting of a holiday emblem, a letter, a number, a sports device, a team emblem, and a flower; and optionally, a secondary aesthetic device configured and dimensioned to be releasably affixed about the body between said ends, and said secondary device being fabricated from a material selected from the group consisting of: permeable, and non-permeable, or both; wherein said material further has the characteristics of being malleably poseable with memory, said secondary device further optionally including a wicking barrier about the orifice, wherein said barrier is selected from the group consisting of: a chemical layer, and a physical layer.

25. A passive fragrance delivery system as in claim 24, wherein the secondary device is one or more leaves.

26. A passive fragrance delivery system as in claim 19, further including a surface cover selected from the group consisting of: a grass, a moss, and a hay.
